(19) United States
(12) Patent Application Publication
(10) Pub. No.: US 2012/0269914 A1
(43) Pub. Date: Oct. 25, 2012

(54) PLANT EXTRACTS FOR TREATING SKIN DISEASES

(76) Inventor: Rudy Susilo, Berlin (DE)

(21) Appl. No.: 13/260,706

(22) PCT Filed: Mar. 29, 2010
(86) PCT No.: PCT/DE2010/000359

§ 371 Date: Jul. 10, 2012

(30) Foreign Application Priority Data
Mar. 27, 2009 (DE) 10 2009 014 834.5

(51) Int. Cl. A61K 36/88; A61P 17/00; A61Q 19/08; A61P 3/00; A61P 17/10; A61K 8/97; A61P 17/06

(52) U.S. Cl. 424/777

(57) ABSTRACT

The invention relates to the utilization of an extract or of extracts, of lipophilic extracts as well as hydrophilic extracts from the fruit of the plant Pandanus conoideus for production of topical preparations for the treatment of diseases of skin and of the mucous membrane in the region of the nose, eyes and/or ears, and in particular for treating of dry skin, neurodermatitis, dermatitis, eczemas, psoriasis, metabolic disorders of the skin, acne, damaged skin, symptoms of skin aging and for smoothing the skin, increasing the skin's hydration and for the treatment and prophylaxis of neurodermatitis, dermatitis, eczemas, and psoriasis.

The invention relates to the utilization of an extract or extracts, of lipophilic extracts as well as hydrophilic extracts from the fruit of the plant Pandanus conoideus for the production of topical formulations and in particular of emulsions for use with dry skin, metabolic disorders of the skin, acne, damaged skin, symptoms of skin aging and for smoothing the skin and increasing the skin's hydration as well as for the treatment and prophylaxis of neurodermatitis, dermatitis, eczemas, psoriasis and of the mucous membrane in the area of nose, ears and eyes.

Neurodermatitis is also called atopic eczema, endogenous eczema, chronic constitutional eczema, asthma eczema, Besnier's prurigo or atopic dermatitis. The disease is chronic and non-contagious. Moreover, it is considered to be treatable but currently still incurable. The disease, which expresses itself in red, dry, exfoliative skin and sometimes oozing eczema on the skin, is in most cases accompanied by severe itching and is usually treated with the topical application of anti-inflammatory substances.

The causes of neurodermatitis have not yet been completely clarified. Experts see the complex incidence of the disease and its very individual development as an interaction of genetic factors, psychic influences, immunological changes and environmental effects. More recent results show that the barrier function of the skin is decisively impaired where atopic dermatitis is present. The skin increasingly loses water, in particular because of disruptions of the lipid barrier function (skin lipid barrier). Therefore, the atopic eczema in particular expresses itself by sensitive and dry skin which is often also reddened. The weakened barrier facilitates the intrusion of allergens and increases the inflammation tendency of atopic skin. The skin is prone to external irritations which might lead to itching.

Until now, no special reliable examinations for diagnosing neurodermatitis are known. The physician typically makes a comprehensive anamnesis in which the medical records and family circumstances are taken into consideration. The following physical examination differentiates between diagnostic criteria of the first and second order, and the diagnosis of neurodermatitis is made as soon as a patient shows at least three features of the first order and at least three features of the second order.

Diagnostic criteria of the first order among others include eczema at places which are extremely typical for neurodermatitis, such as the hollows of the knee, crooks of the arm, neck, throat and face, as well as severe itching, a chronic development of the disease and also incidences of neurodermatitis, hay fever or asthma in the family.

Diagnostic criteria of the second order include but are not limited to itching when sweating, dark eye circles, paleness around the mouth, a congenital Dennie-Morgan line or more pronounced outline of skin lines, positive allergy tests, ichthyosis, xerosis or predisposition to skin infections. In addition to this, neurodermatitis patients show a predisposition to a narrowing of the blood vessels in the area of the skin which expresses itself by a so-called white dermographism where e.g. after scratching, there are no red welts on the skin but white ones.

This cannot be a conclusive enumeration since the causes of neurodermatitis have not been completely clarified and the very complex incidence of the disease has a very individual development. Thus, the diagnosis of neurodermatitis may even be correct if less than three diagnostic criteria are given but these are evaluated as so severe by the physician that a neurodermatitis is considered to be very likely.

Pandanus conoideus is an endemic type of a plant from Irian Jaya, Indonesia. The natives use the fruits of this plant as foodstuff, food supplement, forage, natural colourant and cure for different diseases such as helminthic diseases and blindness.

The traditional preparation takes place by long cooking of the outer red fruit layer and subsequent grinding of the pulp in order to get a mush that is free of pips. The oily mush gained like this is then used as daily foodstuff or a cure.

The Pandanus conoideus is a pumpkin-like oblong fruit with pipless white pale pulp inside and red pulp around this where the pips of the fruit can be found. On the outside, the cylindrical fruit, which is 1 m long, weighs 7.5 kg and has a diameter of 12 cm on average and is thus relatively big, is surrounded by a red skin.

The task of the present invention is to provide a remedy for the application, prophylaxis and treatment of different skin diseases such as dry skin, metabolic disorders of the skin, acne, damaged skin, symptoms of aging of the skin, neurodermatitis, dermatitis, eczemas and psoriasis.

This task is solved with the technical teaching of the independent patent claims. Advantageous embodiments of the invention are described in the dependent patent claims, the description as well as in the examples.

Surprisingly, it was found that the extracts of the species Pandanus conoideus can successfully be used as cosmetic and pharmaceutical remedies for the treatment and prophylaxis in particular of neurodermatitis, dermatitis and eczema as well as for cosmetic application for dry skin, metabolic disorders of the skin, acne, psoriasis, damaged skin and symptoms of aging of the skin as well as for the treatment of the mucous membrane in the area of nose, ears and eyes.

There are different forms and colours of the fruits of Pandanus conoideus, such as oblong red fruits, short red fruits, brown and yellow fruits. All of these fruits can be used for the inventive purposes and utilizations.

Surprisingly, it was found that the inventive pandanus extracts are excellently suited for the treatment of neurodermatitis. In a clinical study with 20 patients suffering from neurodermatitis, a reduction of the average atopy score from 30.79 to 18.04 was already found after application over 14 days. This corresponds to a reduction by −41.40% compared to the initial value. With the application over 21 days, the average atopy score was reduced from 30.79 to 6.4. This corresponds to a reduction by −79.23% compared to the initial value. This success of the treatment could again be increased with the utilization of special formulations such as micro-emulsions or nano-emulsions, for example.

The cosmetic and pharmaceutical pandanus extract preparations are particularly preferably given as emulsions, such as an emulsion of the type water in oil (W/O) or oil in water (O/W). Emulsions are heterogeneous mixtures consisting of two liquids which cannot be mixed with each other, with one of the two liquids found in the other as micro-dispersed small drops.

The term “emulsion” as used here refers to the system of oil drops which are micro-dispersed in a water phase or water drops which are micro-dispersed in an oil phase on the one hand as well as to the finished cosmetic or pharmaceutical formulation for the end user on the other hand.

In order to differentiate the galenic system of the emulsion from the ready-to-use formulation in the form of an emulsion, the term “emulsion formulation” is sometimes also used here for the ready-to-use formulation in the form of an emulsion.

According to the invention, emulsion formulations, ointments, pastes, creams or gels are preferred for the ready-to-use form. These forms of the final products, i.e. these forms for the ready-to-use product preferably contain the system of the emulsion and particularly preferably micro-emulsions or nano-emulsions. This means that preferably, the pandanus extract is dispersed in an oil phase and the oil phase is dispersed in an aqueous phase in the form of small drops. This galenic system of an emulsion may be used as a ready-to-use emulsion formulation in the present form or the emulsion may be an ingredient of a cream, paste, ointment or gel; the cream, paste, ointment or gel are then respectively the ready-to-use final formulations. The use of micro-emulsions or nano-emulsions as a ready-to-use emulsion formulation or as an ingredient of a cream, paste, ointment or gel is particularly preferred. Nano-emulsions are again preferred compared to micro-emulsions.

Micro-emulsions are emulsions which show an average drop size of the oil drops of 1 μm to 50 μm and nano-emulsions are emulsions with an average drop size of the oil drops of 50 nm to 800 nm.

The oil phase of the inventive preparations is selected from the pandanus extract alone or a mixture of pandanus extract with other natural and/or synthetic oils. The other oils are selected from the group of esters from saturated and/or unsaturated, branched and/or unbranched alkane carboxylic acids of a chain length of 3 to 30 c-atoms and saturated and/or unsaturated, branched and/or unbranched alcohols of a chain length of 3 to 30 c-atoms, from the group of esters of aromatic carboxylic acids and saturated and/or unsaturated, branched and/or unbranched alcohols of a chain length of 3 to 30 c-atoms. These ester oils are advantageously selected from the group isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erycyl oleate, erycyl erucate as well as synthetic, semi-synthetic and natural mixtures of such esters.

Other advantageous groups for the oil phase include branched and unbranched hydrocarbons and hydrocarbon waxes, silicone oils, dialkyl ether, saturated or unsaturated, branched or unbranched alcohols as well as fatty acid triglycerides such as the triglycerides of saturated and/or unsaturated, branched and/or unbranched alkane carboxylic acids of a chain length of 8 to 24 c-atoms. Also advantageous are 2-ethylhexyl isostearate, octyldodecanol, isotridecylisononanoate, isoeicosane, 2-ethylhexyl cocoate, C₁₂₋₁₅-alkyl benzoate, caprylic-capric acid triglyceride as well as dicapryl ether. Any mixture of oils and waxes may be combined according to the invention.

The inventive preparations and formulations advantageously contain one or several emulsifiers, preferably from the following groups: phospholipids such as phosphatidylcholine (PC), phosphatidylglycerol (PG), dipalmitoylphosphatidylcholine (DPPC) or the mixture of phospholipids as well as hydrogenated phospholipids. Sucrose esters in combination with glyceryl stearate and glyceryl stearate citrate, glyceryl stearate in combination with ceteareth-20 and/or ceteareth-25, ceteareth-6 in combination with stearyl alcohol, cetyl stearyl alcohol in combination with PEG-40 castor oil and sodium cetyl stearyl sulfate, triceteareth-4 phosphate, glyceryl stearate, sodium cetyl stearyl sulfate, lecithin trilaureth-4 phosphate, laureth-4 phosphate, stearic acid, propylene glycol stearate SE, PEG-25 hydrogenated castor oil, PEG-54 hydrogenated castor oil and/or PEG-6 capric acid/capric acid glycerides, glyceryl oleate in combination with propylene glycol, PEG-9 stearate, PEG-20 stearate, PEG-30 stearate, PEG-40 stearate, PEG-100 stearate, ceteth-2, ceteth-20, polysorbate-20, polysorbate-60, polysorbate-65 and/or polysorbate-100, glyceryl stearate in combination with PEG-100 stearate, glyceryl myristate, glyceryl laurate, PEG-40 sorbitan peroleate, laureth-4, ceteareth-3 and/or isostearyl glyceryl ether, cetyl stearyl alcohol in combination with sodium cetyl stearyl sulfate, laureth-23 and/or steareth-2, glyceryl stearate in combination with PEG-30 stearate, PEG-40 stearate, glycol distearate, PEG-22 dodecyl glycol copolymer, polyglyceryl-2-PEG-4 stearate, ceteareth-12, ceteareth-20, ceteareth-30, methyl glucose sesquistearate, steareth-10 and/or PEG-20 stearate, steareth-2 in combination with PEG-8 distearate, steareth-21, steareth-20, isosteareth-20, PEG-45/dodecyl glycol copolymer, methoxy PEG-22/dodecyl glycol copolymer, PEG-40 sorbitan peroleate, PEG-40 sorbitan perisostearate, PEG-20 glyceryl stearate, PEG-8 beeswax, polyglyceryl-2-laurate, isostearyl diglyceryl succinate, stearamidopropyl-PG-dimonium-chloride phosphate, glyceryl stearate SE, ceteth-20, triethyl citrate, PEG-20 methyl glucose sesquistearate, glyceryl stearate citrate, cetyl phosphate, cetearyl sulfate, sorbitan sesquioleate, triceteareth-4-phosphate, trilaureth-4-phosphate, polyglyceryl methyl glucose distearate, potassium cetyl phosphate, polyglyceryl-3 methyl glucose distearate isosteareth-10, polyglyceryl-2-sesquiisostearate, ceteth-10, oleth-20 and/or cetyl palmitate, cetyl stearyl alcohol in combination with PEG-20 stearate, PEG-30 stearate, PEG-40 stearate and/or PEG-100 stearate, hydrogenated lecithin as well as medium-chain triglycerides. The above mentioned emulsifiers are preferred and may be part of the carrier system.

The preparations and formulations may contain system stabilizing agents and consistency-increasing substances, e.g. cellulose derivates, chitosan derivates, starch derivates, guar gum, agar, carrageenan derivates, alginates and polysaccharides.

In another embodiment, the preparations and formulations are given as a gel which contains additional organic thickening agents, e.g. gum arabic, xanthan gum, sodium alginate, cellulose derivates, preferred methyl cellulose, hydroxymethyl cellulose, hydroxyethyl cellulose, hydroxypropyl cellulose, hydroxypropyl methyl cellulose or anorganic thickening agents such as aluminium silicates, for example bentonite or a mixture of polyethylene glycol and polyethylene glycol stearate or distearate, in addition to the pandanus extract and solvents which are usually used. These substances may as well be parts of the carrier system.

The preparations and formulations may be formulated as gels, ointments, creams, lotions, solutions, pastes, oils, suspensions, powder or semisolid formulations as well as anhydrous preparations. Preferably, the above mentioned formulations contain an emulsion and even more preferably a micro-emulsion or nano-emulsion of the pandanus extract in an aqueous phase or just in water.

Cosmetic and pharmaceutical compositions and formulations contain additives which are known to the experts, including stabilizers, bulking agents, perfume, colourants, emulsifiers, additional active agents such as vitamins, sunscreens, alcohol, water, salts as well as antimicrobial or proteolytic substances which may form the carrier system together with the aqueous phase or water.

The preparations may contain lipid particles in which the active agents of the extract can be transported. The pandanus extract has a regenerative effect on the skin lipid barrier thanks to the high content of phytolipids. A reduction of symptoms can already be achieved with the restoration of the lipid barrier. A cosmetic or pharmaceutical formulation which is an emulsion formulation or contains an emulsion, preferably a micro-emulsion or nano-emulsion, is best suited for the regeneration of the lipid barrier.

Other formulations without skin care additives were also developed and evaluated. Due to the high concentration of phytolipids and the strong anti-inflammatory effect of pandanus extract, only the effect of the extract is able to suppress the skin symptoms of neurodermatitis, namely the itching. Surprisingly, this works all the better the smaller the oil drops containing the pandanus extract in an emulsion are.

The treatment of neurodermatitis is mostly effected with the step-by-step therapy:

1st step: utilization of fatty humectant substances (emollients) as a basic therapy in order to regenerate the skin lipid barrier.
2nd step: utilization of anti-inflammatory substances such as corticosteroids in the flare-up phase in order to suppress the release of inflammation factors.
3rd step: in case of a high degree of severity of the diseases, calcineurin inhibitors such as tacrolimus may be used.

The present results however surprisingly show an unexpectedly strong anti-inflammatory effect of the extract. The IC-50 values for the suppression of the inflammation factors TNF-α, IL-6 and PGE-2 amount to <10 ug/ml for IL-6 and TNF-alpha and/or <50 ug/ml for PGE-2 (FIGS. 3-5). The pandanus extract thus has a dualistic effect since the extract restores the skin lipid barrier and strongly suppresses the inflammation processes. Insofar, the mechanisms of action of the extract are similar to the recommended therapy scheme for the treatment of immunodermatologic diseases. This unexpectedly strong and dualistic mechanism of action of the pandanus extract was surprising and results in a clearly better effect with dermatologic and immunodermatologic diseases than the experts had expected and also in a considerably better effect than similar extracts which are used for skin care and skin regeneration, such as orchid extract, rose extract, witch hazel extract, aloe vera extract, tonka extract, spelt extract or similar. In examinations of subjects with dry skin, the pandanus extract came off best compared to all the other extracts mentioned above according to subjective estimations and impressions of the subjects after utilization over three weeks and several times a day. All the above mentioned extracts were produced as a cream in the same concentration according to formulation 6 and tested by application and embrocation of approx. 1 ml cream per 4 cm² of dry skin surface.

The inventively used extracts can be obtained in the known way, for example
1) by direct pressing of the red pulp from which a product containing oil or an oily product is obtained
2) by extraction of the red pulp with organic solvents and in particular lipophilic solvents from which a product containing oil or an oily product is obtained, too
3) by alcoholic extraction of the red pulp
4) by alcoholic extraction of the pips
5) by direct pressing or alcohol extraction of the red pulp together with the pips
6) by direct pressing of the pale inner part of the fruit from which aqueous juice is obtained or
7) by alcoholic extraction of the dried pale inner part of the fruit from which hydrophilic extracts are obtained.

These extracts can be used in the pure form or processed as a cosmetic remedy for utilization in cases of dry skin, metabolic disorders of the skin, acne, damaged skin, symptoms of aging and for smoothing the skin and increasing the skin's hydration.

Moreover, these extracts can be used in the pure form or processed as a pharmaceutical remedy for the treatment and prophylaxis of neurodermatitis, dermatitis, eczemas, metabolic disorders of the skin, acne and damaged skin.

Upon use of the extracts of the species Pandanus conoideus, no undesirable side effects could be found and the extracts are thus classified as being very well tolerated and suited for long-term utilization such as in the case of neurodermatitis, for example.

Thus, the present invention relates to the utilization of an extract gained from the species Pandanus conoideus for the production of a topical formulation without or with a carrier system for the treatment and prophylaxis of diseases of the skin and mucous membrane in the area of the nose, eyes and/or ears as well as for smoothing the skin and increasing the skin's hydration. The diseases of the skin are mainly dry skin, neurodermatitis, dermatitis, eczemas, psoriasis, metabolic disorders of the skin, acne, damaged skin and symptoms of aging. If the topical formulation contains a carrier system, the topical formulation contains a carrier system in the form of an oil-in-water emulsion or of a water-in-oil emulsion and preferably in the form of an oil-in-water emulsion.

In this, the term “carrier system” includes any components which have not been obtained by extraction from the species Pandanus conoideus. Thus, any ingredients, including water, which are not part of the pandanus extract are principally designated as the carrier system. Preferably, the pandanus extract is dissolved, emulsified and/or dispersed in a liquid, semisolid or solid carrier system. Examples for suitable carrier systems are liquids such as water or aqueous buffer solutions, physiologically well-tolerated organic solvents such as ethanol or combinations thereof, oil-water emulsions, water-oil emulsions, fats, polyethylene glycols, propylene glycols, glycerin, emulsifiers or combinations thereof as well as other carrier substances or additives such as penetration enhancers and emulsifiers which are used in pharmaceutical and cosmetic formulations. Such carrier systems preferably arrange for a targeted release of active agents to the skin cells and/or modulate the absorption by the skin cells, which helps to achieve a longer effect of the pandanus extract. In particular, a stable formulation is obtained with the formulation with a carrier system which is particularly advantageous for topical preparations for the treatment of skin diseases.

In another embodiment, the invention relates to the utilization of an extract gained from the species Pandanus conoideus for the production of topical preparations for the treatment and prophylaxis of diseases of the skin and mucous membrane in the area of the nose, eyes and/or ears as well as for smoothing the skin and increasing the skin's hydration.

The inventive topical preparation advantageously contains one or several penetration enhancers. In this, the term “penetration enhancer” covers any substances which bring about an improved penetration of active agents by the skin regardless of their mode of action.

The penetration enhancers are selected from the group of fatty alcohols such as aliphatic alcohols, decanol, lauryl alcohol (dodecanol), linoleyl alcohol, nerolidol, 1-nonanol, n-octanol, oleyl alcohol, also from the group of acid esters such as acetic acid-n-butyl ester, cetyl lactate, decyl N,N-dimethylamino acetate, decyl N,N-dimethylamino isopropionat, diethylene glycol oleate, diethyl sebacate, dodecyl N,N-dimethylamino acetate, dodecyl (N,N-dimethylamino)-butyrate, deodecyl N,N-dimethylamino isopropionat, dodecyl 2-(dimethylamino) propionate, EO-5-oleyl ester, ethyl acetate, ethyl acetoacetate, ethyl propionate, glycerol monoether, glycerol monolaurate, glycerol monolinoleate, isopropyl isostearate, isopropyl linoleate, isopropyl myristate, isopropyl myristate/fatty acid glyceride monoglyceride combination, isopropyl myristate/ethanol/L-lactic acid (87:10:3) combination, isopropyl palmitate, methyl acetate, methyl caprate, methyl laurate, methyl propionate, methyl valerate, 1-monocaproyl glycerol, monoglyceride (medium chain), nicotinic ester (benzyl), octyl acetate, octyl N,N-dimethylamino acetate, oleyl oleate, n-pentyl N-acetyl prolinate, propylen glycol monolaurate, sorbitan dilaurate, sorbitan dioleate, sorbitan monolaurate, sorbitan monooleate, sorbitan trilaurate, sucrose coconut fatty acid mixtures, sucrose monolaurate, sucrose monooleate, tetradecyl N,N-dimethylamino acetate.

Also advantageous are penetration enhancers from the group of fatty acids such as alkanoic acids, capric acids, binary acids, ethyl octadecanoic acids, hexanoic acids, lactic acids, lauric acids, linoelaidic acids, linoleic acids, linolenic acids, neodecanoic acids, pelargonic acids, propionic acids as well as vaccenic acids; other advantageous penetration enhancers can be found in the group of fatty acid esters such as α-monoglycerol ether, EO-2-oleyl ether, EO-5-oleyl ether, EO-10-oleyl ether, ether derivates of polyglycerol and alcohols (1-O-dodecyl-3-O-methyl-2-O-(2′, 3′-dihydroxypropyl (glycerol)). Also advantageous are L-α-amino acids, lecithin, phospholipids, saponin/phospholipids, sodium deoxycholate, sodium taurocholate, sodium tauroglycocholate, acid phosphatases, calonases, orgelses, papain, phospholipase A-2, phospholipase C, triacylglycerol hydrolase, acetamide derivates, acyclic amides, N-adamantyl, n-alkanamide, clofibric acid amide, N,N-didodecyl acetamide, di-2-ethyl hexyl amine, diethyl methyl benzamide, N,N-diethyl-m-toluamide, N,N-dimethyl-m-toluamide, ethomeen S12 [bis-2(2-hydroxyethyl)oleyl amine], hexamethylene lauramide, laurylamine (dedecylamine), octyl amide, oleyl amine, unsaturated cyclic ureas, urea, β and γ-cyclodextrin complexes, hydroxypropyl methylcellulose, liposomes, naphthalene diamide diimide, naphthalene diester diimide, macro-cyclic lactone, ketones and anhydrids.

From the group of surfactants, the use of penetration enhancers is particularly advantageous, such as polyoxyethylene-(4)-lauryl ether, polyoxyethylene-(23)-lauryl ether, polyoxyethylene-(2)-cetyl ether, polyoxyethylene-(10)-cetyl ether, polyoxyethylene-(20)-cetyl ether, polyoxyethylene-(2)-stearyl ether, polyoxyethylene-(20)-stearyl ether, polyoxyethylene-(2)-oleyl ether, polyoxyethylene-(20)-oleyl ether, cetyl trimethyl ammonium bromide, empicol ML26/F, HCO-60, hydroxy polyethoxy dodecan, ionic surfactants (ROONa, ROSO₃Na, RNH₃Cl, R=8-16), lauroyl sarcosine, nonoxynol, octoxynol, phenyl sulfonate, pluronic F127, pluronic L62, pluronic F68 (polyoxyethylene-polyoxypropylene block copolymer), poly oleate, rewopal HV10, sodium laurate, sodium lauryl sulfate (sodium dodecyl sulfate), sodium oleate, span 20, span 40, span 85, synperonic NP, triton X-100, tween-20, tween-40, tween-60, tween-80 and tween-85.

Also advantageous are aliphatic thiols, alkyl N,N-dialkyl-substituted amino acetate, anise oil, anticholinergic substances, ascaridol, two-phase derivates, bisabolol, cardamom oil, 1-carvone, chenopodium oil, 1,8 cineole (eucaplyptus oil), codliver oil (fatty acid extract), 4-decyloxazolidin-2-one, dicyclohexylmethylamine oxide, diethyl hexadecyl phosphonate, diethyl hexadecyl phosphoramidate, N,N-dimethyldodecyclamine-N-oxide, 4,4-dimethyl-2-undecyl-2-oxazolin, N-dodecanoyl-L-amino acid methyl ester, 1,3-dioxycycloalkan (SEPAs), dithiothreitol, eucalyptol (cineol), eugenol, herbal extracts, lactam N-acetic acid ester, N-hydroxyethyl acetamide, 2-hydroxy-3-oleoyloxy-1-pyroglutamyl oxypropane, menthol, menthon, morpholine derivates, N-oxides, nerolidol, octyl-β-(thio)glucopyranoside, oxazolidinone, piperazine derivates, polar lipids, polydimethlysiloxane, poly [2-(methylsulfinyl)ethyl acrylate], polyrotaxane, polyvinyl benzyl dimethyl ammonium chloride, poly (N-vinyl-N-methyl acetamide), prodrugs, saline solutions, sodium pyroglutaminate, terpenes and azacyclo ring compounds, vitamin E (α-tocopherol) as well as ylang ylang oil.

Also advantageous are penetration enhancers from the group of the N-methylpyrrolidones such as N-cyclohexyl-2-pyrrolidone, 1-butyl-3-dodecyl-2-pyrrolidone, 1,3-dimethyl-2-imidazolikinone, 1,5 dimethyl-2-pyrrolidone, 4,4-dimethyl-2-undecyl-2-oxazoline, 1-ethyl-2-pyrrolidone, 1-hexyl-4-methyloxycarbonyl-2-pyrrolidone, 1-hexyl-2-pyrrolidone, 1-(2-hydroxyethyl)pyrrolidone, 3-hydroxy-N-methyl-2-pyrrolidinone, 1-isopropyl-2-undecyl-2-imidazoline, 1-lauryl-4-methyloxycarbonyl-2-pyrrolidone, N-methyl-2-pyrrolidone, poly (N-vinyl pyrrolidone, pyroglutamic acid ester and 2-pyrrolidone (2-pyrrolidinone)).

From the group of ionic compounds, the use of penetration enhancers is particularly advantageous such as ascorbate, calcium thioglycolate, cetyl trimethyl ammonium bromide, 3,5-diiodosalicylate sodium, lauroyl choline iodide, 5-methoxysalicylate sodium, monoalkyl phosphate, 2-PAM chloride, 4-PAM chloride (derivates of N-methylpicolinium chloride, sodium carboxylate, sodium hyaluronate, sodium lauryl sulfate (sodium didecyl sulfate)) and also dimethyl sulfoxide and related substances such as cyclic sulfoxides, decymethyl sulfoxide and 2-hydroxyundecyl methyl sulfoxide.

The penetration enhancers are furthermore selected from the group of solvents and related substances such as acetamides, acetone, n-alkanes (chain length between 7 and 16), alkanols, diols, short-chain fatty acids, cyclohexyl-1,1-dimethyl ethanol, dimethyl acetamide, dimethyl formamide, ethanol, ethanol/d-limonen combinations, 2-ethyl-1,3-hexanediol, ethoxydiglycol, glycerol, glycol, lauryl chloride, limonene N-methyl formamide, 2-phenyl ethanol, 3-phenyl-1-propanol, 3-phenyl-2-propen-1-ol, polyethylene glycol, polyoxyethylene sorbitan monoester, polypropylene glycol, tridecanol, propylene glycol, span 20, squalene, triacetin, trichloroethanol, trifluoroethanol, trimethylene glycol, xylene.

Another advantageous group of penetration enhancers includes the azons such as N-acyl-hexahydro-2-oxo-1H-azepin, N-alkyl-dihydro-1,4-oxazepin-5,7-dion, N-alkyl morpholine-2,3-dion, N-alkyl morpholine-3,5-dion, aza-cycloalkan derivates (ketones and thiones), aza-cycloalkenone derivates, 1-[2-(decylthio)ethyl]aza-cyclopentan-2-one (HPE-101), N-(2,2-dihydroxyethyl)dodecylamine, 1-dodecanoylhexahydro-1-H-azepin, 1-dodecyl aza-cycloheptan-2-one (azon or laurocapram), N-dodecyl diethanolamine, N-dodecyl-hexahydro-2-thio-1H-azepin, N-dodecyl-N-(2-methoxyethyl)acetamide, N-dodecyl-N-(2-methoxyethyl)isobutyramide, N-dodecylpiperidin-2-thione, N-dodecyl-2-piperidinone, N-dodecylpyrollidin-3,5-dion, N-dodecylpyrollidin-2-thione, N-dodecyl-2-pyrrolidon, 1-farnesyl aza-cycloheptan-2-one, 1-farnesyl aza-cyclopentan-2-one, 1-geranyl aza-cycloheptan-2-one, 1-geranyl aza-cyclopentan-2-one, hexahydro-2-oxo-azepine-1-acetic acid ester, N-(2-hydroxyethyl)-2-pyrrolidon, 1-lauryl aza-cycloheptan, 2-(1-nonyl)-1,3-dioxolan, 2-(1-nonyl)-1,3-dioxolan, 1-N-octyl aza-cyclopentan-2-one, N-(1-oxododecyl)-hexahydro-1H-azepin, N-1 (1-oxododecyl)-morpholine, 1-oxohydrocarbyl-substituted aza-cyclohexans, N-(1-oxotetradecyl)-hexahydro-2-oxo-1H-azepin as well as N-(1-thiododecyl)-morpholine. All the aforementioned penetration enhancers may be part of the carrier system.

In addition, the present invention also relates to the utilization of extracts of Pandanus conoideus for the production of a pharmaceutical remedy for the treatment and prophylaxis of neurodermatitis, dermatitis, eczemas, metabolic disorders of the skin, acne, psoriasis and damaged skin. The formulations used as a pharmaceutical remedy may also serve as cosmetic compounds and be used in the case of dry skin, symptoms of aging of the skin and for skin hydration.

Moreover, the present application discloses processes for obtaining the extracts, such as a lipophilic extract from the red pulp of the fruits of the species Pandanus conoideus which is obtained by direct pressing, i.e. cold-pressing of the separated red pulp, preferably without any chemical additives at temperatures preferably below 40° C. or by extraction of the separated ground red pulp with organic solvents at temperatures preferably below 40° C. Extractions may principally be carried out at temperatures between 4° C. and 100° C. However, cold-pressing below 40° C. is particularly preferred since on the one hand, it was found that important bioactive substances such as tocopherols and other vitamins, phytosterols etc. are destroyed when the red pulp is boiled in water or an organic solvent. Moreover, an extract produced by means of boiling turns rancid very quickly. By means of the preferred cold-pressing, surprisingly one gets extracts, mainly oils, which are stable for at least one year and probably considerably longer. Stability dates until now show a stability of two years of samples which have until now been stored at 4° C. for two years. The stability test is being continued and considerably longer stability values are expected.

A temperature increase during the extraction procedure or processing procedure of the red pulp above 40° C. surprisingly leads to a reduction of the stability and storage stability of the extract obtained. The longer the temperature is above 40° C. during the extraction of the red pulp and the higher the temperature is above 40° C. during the oil production process from the red pulp, the lower the stability of the extract. Thus, it is important to take care that the extraction and processing conditions do not exceed a temperature of 60° C. or preferably 50° C. and particularly preferably 40° C. during the complete production process of the oil from the red pulp and that in the ideal case, the complete process of extraction and oil production is carried out at room temperature.

For the extraction, preferably hexane, heptane, cyclohexane, chloroform, methylene chloride, acid ester, diethyl ether, petroleum ether, tert-butyl methyl ether, THF, methanol, ethanol, propanol, isopropyl, butanol, isobutanol, sec-butanol, acetone and mixtures of these solvents are used. After extraction, the solvent is preferably removed and recycled in order to be re-used for further extractions. The concentrated oil contains the parts of the extracted pulp which have an effect on the weight.

Another preferred extraction procedure is an alcohol and/or alcohol-water extraction of the pulp with the pips in which first, the red pulp and pips are ground at temperatures preferably below 40° C., then mixed with alcohol and/or an alcohol-water mixture while stiffing and then squashed and filtered. Prior to grinding, the pulp and pips can also be dried. The ground pips together with the ground pulp are then afterwards treated analogously to the procedure for alcohol and/or alcohol-water extraction of the fresh material.

Another procedure of the present application refers to the production of a hydrophilic extract from the separated pips of the fruit of the species Pandanus conoideus by crushing the pips at temperatures preferably below 40° C. and extraction of the crushed pips with a hydrophilic solvent at temperatures preferably below 40° C. As with the other extractions, here, the extractions may again in principle be carried out at temperatures between 4° C. and 100° C. However, temperatures of preferably 60° C. and particularly preferably temperatures of 40° C. should not be exceeded.

As hydrophilic solvents, preferably water, acetone, tetrahydrofuran (THF), methanol, ethanol, propanol, isopropyl, butanol or mixtures of these solvents are used, but water, methanol and ethanol or mixtures of water, methanol and ethanol are preferred. After a possibly multiple extraction and mixing of the hydrophilic extracts, the solvent is removed and re-used. These steps should preferably be carried out at temperatures below 40° C.

Another procedure of the present application relates to the production of a hydrophilic extract from the white pulp of the fruits of the species Pandanus conoideus by direct pressing of the separated white pulp with or without additives, such as the addition of antioxidants at temperatures preferably below 40° C. or by drying and crushing the separated white pulp at temperatures preferably below 40° C. and subsequent extraction of the dried crushed white pulp with hydrophilic solvents.

As hydrophilic solvent, preferably water, acetone, tetrahydrofuran (THF), methanol, ethanol, propanol, isopropyl, butanol or mixtures of these solvents are used, but water, methanol and ethanol or mixtures of water, methanol and ethanol are preferred. After a possibly multiple extraction of the white and/or pale pulp and mixture of the hydrophilic extracts, the solvent is removed and can be re-used. All the extraction steps should preferably be carried out at temperatures preferably below 40° C. The concentrated extract contains the pharmacologically active ingredients of the white pulp of the Pandanus conoideus.

The fruit of the Pandanus conoideus consists of an inner pale fruit part and a red outer fruit part containing pips and oil. The present application discloses 7 possible extracts which can be obtained from this fruit. These extracts can be used individually or combined, preferably as an aqueous solution, as an oil or as a solid.

For obtaining the bioactive extracts, the fruits are cleaned and the red outer fruit layer is separated from the white, pale inner fruit part. The different extracts can be obtained in different ways as it is for example disclosed in the examples 1-7.

All the steps of the procedure are preferably carried out at temperatures between 5 and 80° C., more preferably at 10° C. to 60° C., more preferably at 15-45° C., even more preferably at 17-40° C. and particularly preferably at 19° C.-38° C. In addition to this, it is also preferred that the steps of the procedure are carried out at temperatures below 60° C., preferably below 50° C. and particularly preferably below 40° C.

A simplified procedure for obtaining an extract from the red pulp consists in first separating the red pulp from the pips, then grinding it and extracting with an organic solvent several times. If ethanol is used for the extraction, this alcoholic extract can directly be used as a pharmaceutical or cosmetic remedy. This procedure is particularly suited for the use of physiologically tolerated solvents and preferably for alcohols such as ethanol, isopropyl and particularly for ethanol.

A preferred procedure for obtaining an extract from the red pulp together with the pips consists in first grinding the red pulp with the pips and then extracting several times with an organic solvent. If ethanol is used for the extraction, this alcoholic extract can directly be used as a pharmaceutical or cosmetic remedy. This procedure is particularly suited for the use of physiologically tolerated solvents and preferably for alcohols such as ethanol, isopropyl and particularly for ethanol. The extract can of course also be produced from the dried red pulp with pips with the same procedure.

A simplified procedure for obtaining an alcoholic extract from the pale pulp is to dry the pale white inner pulp, preferably in air, then to grind it and extract several times with a hydrophilic solvent. If ethanol or ethanol-water mixtures are used for the extraction, this alcoholic extract can directly be used as medication.

Another possibility is to cold-press the inner pale pulp (preferably T<40° C.). The aqueous extract obtained by this can then be used in the given form as a pharmaceutical or cosmetic remedy.

If the extraction of the separated and crushed pips takes place with ethanol, such ethanolic extract can also directly be used as a pharmaceutical or cosmetic remedy.

Thus, the present invention particularly also relates to ethanolic extracts which were directly obtained from the red, the pale and/or the white pulp as well as from the pips under gentle conditions (preferably T<40° C.). The alcoholic solution obtained can be concentrated and serve for the preparation of an ethanolic solution or solid after drying, and the solid can be added to common formulations as an active component.

A preferred extraction procedure for Pandanus conoideus consists for example of the following steps:
1) Division of the fruit into pale, white inner pulp and the red layer where the red pulp and pips are located.
2) Cold-pressing or extraction of the red layer with organic solvents in order to obtain the oily fraction and
3) Division of the red layer into red pulp and pips.
4) Cold-pressing of the red pulp in order to obtain the oil fraction and
5) Extraction of the residue of the red pulp with alcohol (e.g. methanol, ethanol, propanol, isopropyl, butanol); or
6) Extraction of the oil directly from the red pulp with alcohol or other organic solvents,
7) Extraction of the pips with hydrophilic solvents,
8) Cold-pressing of the white pale pulp in order to obtain juice, or
9) Optional extraction of the white pale pulp with hydrophilic solvents and/or
10) Drying of the white pale pulp and
11) Subsequent extraction of the white pale pulp with hydrophilic solvents.

Similar extraction procedures can be described as follows and include at least some of the following steps:
a) Division of the fruit in pale white inner pulp and red pulp with the pips,
b) Separation of the red pulp from the pips,
c) Cold-pressing or extraction of the red pulp together with the pips with organic solvents,
d) Cold-pressing of the red pulp and
e) Crushing and/or homogenization of the red pulp, and
f) Extraction of the juice from the red pulp as well as of the crushed or homogenized red pulp with alcohols or other organic lipophilic solvents, and
g) Collection and concentration of the lipophilic extracts, or
h) Crushing of the pips and
i) Extraction of the crushed pips with hydrophilic solvents and
j) Collection and concentration of the hydrophilic extracts, or
k) Cold-pressing of the white pale inner pulp or crushing and/or homogenization of the dried white pale pulp, and
l) Extraction of the crushed or homogenized dried white pale pulp with hydrophilic solvents and
m) Collection and concentration of the hydrophilic extracts.

An extract or several extracts from the Pandanus conoideus is/are preferably used for the production of a pharmaceutical, dermatological or cosmetic compound which contains the extract or extracts from the Pandanus conoideus together with at least one pharmacologically or cosmetically tolerated additive, excipient and/or solvent.

The present application discloses procedures according to which different extracts can be obtained from the fruit of the plant Pandanus conoideus, depending on the type of extraction carried out. It must be noted, of course, that red pulp and pale white pulp may also be jointly extracted and preliminary separation is not obligatory. The same also applies to the red pulp and pips. These pandanus extracts all show a similar pharmacological and cosmetic activity, i.e. all the extracts are equally suited for the prophylaxis and treatment of neurodermatitis, dermatitis, eczemas, psoriasis, metabolic disorders of the skin, acne and damaged skin.

Even with regard to the cosmetic applications, all pandanus extracts show a similar effectiveness, in particular in the case of dry skin, metabolic disorders of the skin, acne, damaged skin, symptoms of aging of the skin, wrinkles as well as for smoothing the skin and increasing the skin's hydration.

Thus, it is inventively preferred that two or even more extracts are mixed or combined and used for the treatment of the above-mentioned indications or for cosmetic application. Consequently, it is preferred that an extract from the red pulp is mixed with an extract from the pips and/or an extract from the pale white pulp.

An extract from the pips can be produced in exactly the same way with an extract from the pale white pulp and/or with the juice after cold-pressing the pale white pulp and/or with an extract from the red pulp and/or with an extract from the juice of the red pulp obtained after cold-pressing and/or the ingredients of the juice after drying it.

An extract from the white dried pale pulp and/or from the juice obtained by cold-pressing the white pale pulp can be combined with an extract from the pips and/or an extract from the red pulp and/or with an oil obtained by cold-pressing the red pulp and/or the ingredients of the oil after drying it.

With such combinations, the efficacy spectrum of the combination of extracts and/or of the pharmaceutical and cosmetic remedy containing such combinations of extracts can be broadened and the activity can partially even be increased.

The production of the extracts from the red pulp or from the separated pips or the red pulp with the pips or from the pale inner part of the fruit can also be achieved by extraction with supercritical fluids (carbon dioxide, propane, butane), preferably with carbon dioxide, with or without the additional use of entrainers (methanol, ethanol). With the aforementioned fluids, preferably lipophilic extracts can be obtained, but less lipophilic and rather polar to hydrophilic extracts can also be obtained upon simultaneous use of polar entrainers. Thus, no polar entrainers are preferably used for extraction of the red pulp while the use of polar entrainers is preferred for extraction of the pips and of the inner pale pulp.

The extracts obtained from the extraction with supercritical fluids (carbon dioxide, propane, butane), preferably with carbon dioxide, can of course also be used individually or in combination with each other and also in combination with extracts obtained without supercritical fluids. For obtaining the extracts, preferably the following extraction procedures are used: ethanol-water extraction, hexane extraction and supercritical CO₂ extraction.

The term “extract” is meant to cover the liquid phase after single extraction as well as the combined liquid phases of several extractions. In addition to this, the term “extract” is also meant to cover the concentrated liquid phase in which the solvent has been partially, mostly or completely removed so that “extract” may also refer to a solid residue which has been obtained after removal of the solvent, preferably under reduced pressure, and after drying of the solid parts, e.g. a lyophilisate.

The extracts described herein can be used individually or in combination with other inventive extracts and/or in combination with other active agents such as anti-inflammatory active agents as they were obtained or in the form of pharmaceutical or cosmetic formulations and compounds for the treatment and prophylaxis of neurodermatitis, dermatitis, eczema, psoriasis, dry skin, metabolic disorders of the skin, acne and damaged skin or for cosmetic application in the case of dry skin, metabolic disorders of the skin, acne, damaged skin, symptoms of aging of the skin, wrinkles as well as for smoothing the skin and increasing the skin's hydration.

The pharmaceutical or cosmetic remedy which contains an extract or several extracts from Pandanus conoideus can be produced with the common solid or liquid excipients or diluents and the usually used pharmaceutical additives according to the respectively desired type of application with a suited dosage in the known way. Such administration forms are for example tablets, mini-tablets, micro-tablets, film-coated tablets, layered tablets, dragées, capsules, micro-capsules, micro-pellets and pellets, pills, granules, powders, solutions, drops, juices, suspensions, suppositories, clysmata, emulsions, dispersions, creams, gels, ointments, syrups or depot forms. Preferred are topical formulations such as ointments, gels, creams, powder, emulsions and the like.

Such preparations are among others suited for intravenous, intraperitoneal, intramuscular, subcutaneous, oral, rectal, transdermal, topical, intradermal, intragastric, intracutaneous, intranasal, intrabuccal, percutaneous or sublingual administration, with, of course, topical utilization being preferred.

The preferred formulations for external or topical utilization may contain additional ingredients such as lecithin, virgin oils, glycerin, ethylene glycol, 1,3-propandiol, tert-butyl alcohol, propylene glycol, polyethylene glycol, benzyl alcohol, benzyl acetone, benzaldehyde, phenyl ethyl alcohol, 2-phenyl acetate, benzyl alcohol, phenyl ethyl alcohol and purified water in addition to the extract or the extract mixture from the species Pandanus conoideus.

Moreover, the formulations described herein may also be complemented with common odourants and/or perfumes, colourants, preservatives, viscosity-increasing substances and/or UV filters. These additives particularly help to increase the colour, light and/or perfume stability of the formulations and/or the storability or consistency. Such additives are contained in the cosmetic or pharmaceutical formulation in total with no more than 10% by weight, preferably 8% by weight, more preferably 6% by weight and particularly preferably 4%.

Cosmetic and/or dermatological formulations particularly refer to skin creams, body lotions, emulsions, ointments, gels, oils as well as balms and any other formulations suited for topical use.

Oral formulations such as tablets can, for example, be produced by mixing the extract(s) with known additives, for example inert diluents such as dextrose, sugar, sorbitol, mannite, polyvinylpyrrolidone, disintegrants such as corn starch or alginic acid, binders such as starch or gelatine, lubricants such as magnesium stearate or talcum.

Accordingly, dragées can be produced by coating pips which are produced analogously to the tablets with mediums that are typically used in dragée coatings, such as polyvinylpyrrolidone or eudragit, shellac, gum arabic, talcum, titanium dioxide or sugar. The dragée shell may then even consist of several layers; the additives mentioned above in relation to the tablets may be used, however.

Solutions or suspensions with the extract can additionally contain mediums improving the taste such as saccharine, cyclamate or sugar as well as aromatic substances such as vanillin or orange extract. They may also contain suspension additives such as sodium carboxymethyl cellulose or preservatives such as p-hydroxybenzoate. Capsules containing the extract can for example be produced by mixing the active agent with an inert carrier such as lactose or sorbitol and then encapsulating it in gelatine capsules.

The oil-containing or oily extract can as well be directly processed to soft gelatine capsules.

As pharmacologically tolerated carriers, lactose, starch, sorbitol, sucrose, cellulose, magnesium stearate, dicalcium phosphate, calcium sulfate, talc, mannitol, ethyl alcohol and the like can be used, for example. Powder as well as tablets may consist of such a carrier by 5 to 95%.

In addition to this, disintegrants, colourants, flavourings and/or binders may be added to the pharmaceutical or cosmetic remedies and compounds.

Moreover, it is preferred that the extracts are combined with pharmaceutical, cosmetic remedies as well as with anti-inflammatory active agents such as alclofenac, aceclofenac, sulindac, tolmetin, etodolac, fenopren, thiaprofen acid, meclofenamic acid, meloxicam, tenoxicam, lornoxicam, nabumetone, acetaminophene, phenacetin, ethenzamide, sulpyrine, mefenamic acid, flufenamic acid, diclofenac sodium, loxoprofen sodium, phenylbutazone, indomethacine, ibuprofen, ketoprofen, naproxen, oxaprozin, flurbiprofen, fenbufen, pranoprofen, floctafenin, piroxicam, epirizol, tiaramide hydrochloride, zaltoprofen, gabexat mesilat, camostat mesilat, ulinastatin, colchicine, probenecid, sulfinpyrazone, benzbromarone, allopurinol, salicylic acid, atropine, scopolamine, levorphanol, ketorolac, tebufelone, tenidap, clofezone, oxyphenbutazone, prexazone, apazone, benzydamine, bucolome, cinchophene, clonixin, ditrazole, epirizol, fenoprofen, floctafenin, glafenin, indoprofen, niflumic acid, suprofen, bufexamac, dexamethasone, hexestrol, methimazole, betamethasone, triamcinolone, fluocinonide, prednisolone, methylprednisolone, hydrocortisone, fluorometholone, beclomethasone dipropionate, estriol, clobetasol, diflorasone diacetate, halbetosal propionate, amcinonid, desoximetasone, halcinonide, mometasone furoate, fluticasone propionate, flurandrenolide, clocortolone, prednicarbate, alclometasone dipropionate or desonide.

Even vitamins such as vitamin A, vitamin C (ascorbic acid), vitamin D, vitamin H, vitamin K, vitamin E, vitamin B1, vitamin B2, vitamin B3, vitamin B5, vitamin B6, vitamin B12, thiamin, riboflavin, niacin, pyridoxine and folic acid can be added to the compound.

Liquid formulations include solutions, suspensions, sprays and emulsions.

Capsules are for example made of methyl cellulose, polyvinyl alcohols or denaturated gelatine or starch.

As disintegrants, starch, sodium carboxymethyl starch, natural and synthetic gums such as carob bean gum, gum karaya, guar gum, astragalus gum and agar as well as cellulose derivates such as methyl cellulose, sodium carboxymethyl cellulose, micro-crystalline cellulose as well as alginates, bolus alba and bentonites can be used. These ingredients can be utilized in amounts of 2 to 30% by weight.

As binders, sugar, starch from grains, rice or potatoes, natural sugars, natural and synthetic gums such as gum acacia or guar gum, gelatine, astragalus gum, alginic acid, sodium alginate, ammonium calcium alginate, methyl cellulose, carboxymethyl cellulose, polyethylene glycol, waxes, sodium carboxymethyl cellulose, hydroxypropyl methyl cellulose, polyvinylpyrrolidon as well as anorganic compounds such as magnesium aluminium silicates can be added. The binders can be added in amounts of 1 to 30% by weight.

As lubricants, stearates such as magnesium stearate, calcium stearate, potassium stearate, stearic acid, highly melting waxes as well as water-soluble lubricants such as sodium chloride, sodium benzoate, sodium acetate, sodium oleate, polyethylene glycol and amino acids such as leucine can be used. Such lubricants can be used in amounts of 0.05 to 15% by weight.

Particularly preferred are micro-emulsions and nano-emulsions which can be formulated as emulsion, ointment, cream or even gel.

“Micro-emulsion” as used herein refers to an oil-in-water emulsion, with the extract from the Pandanus conoideus preferably in the oily phase and the oil drops having an average diameter of 1 μm to 50 μm. The micro-emulsions can also be produced according to known procedures and with known additives as it has also been described in the present application. Formulations like this are surprisingly better suited for topical application than emulsions with larger drop diameters of more than 200 μm or emulsions with a drop diameter of 20 μm to 400 μm. The drop size is set by the type and intensity of the intermixture of the oil phase in the water phase.

Thus, micro-emulsions with an average diameter of oil drops in the micro-emulsion of 1 μm to 50 μm, preferably 5 μm to 40 μm and more preferably 10 μm to 30 μm are particularly suited for the treatment of diseases of the skin and mucous membrane in the area of the nose, eyes and ears as well as in particular for the treatment and prophylaxis of dry skin, neurodermatitis, dermatitis, eczema, psoriasis, metabolic disorders of the skin, acne, damaged skin and symptoms of aging as well as for smoothing the skin and increasing the skin's hydration.

“Nano-emulsion” as used herein refers to an oil-in-water emulsion, with the extract from the Pandanus conoideus preferably in the oily phase and the oil drops having an average diameter of 50 nm to 800 nm. The nano-emulsions can be produced according to known procedures and with known additives as it has also been described in the present application. Formulations like this are surprisingly better suited for the topical application than emulsions with larger drop diameters of more than 200 μm.

Thus, nano-emulsions with an average diameter of oil drops in the micro-emulsion of 50 nm to 800 nm, preferably 100 nm to 600 μm and more preferably 200 nm to 400 nm are particularly suited for the treatment of diseases of the skin and mucous membrane in the area of the nose, eyes and ears as well as in particular for the treatment and prophylaxis of dry skin, neurodermatitis, dermatitis, eczema, psoriasis, metabolic disorders of the skin, acne, damaged skin and symptoms of aging as well as for smoothing the skin and increasing the skin's hydration.

Drop sizes below 1 μm were determined based on their zeta potentials by means of a Nicomp device of Particle Sizing System Inc. For larger drops (1 μm to 200 μm), optical analyses of individual particles were carried out with a PSS Accusizer device.

This preferred suitability of micro-emulsions and nano-emulsions with average drop sizes of 50 nm to 50 μm in particular for the treatment and prophylaxis of dry skin, neurodermatitis, dermatitis, eczema, psoriasis, metabolic disorders of the skin, acne, damaged skin and symptoms of aging is surprising since such formulations are evidently suited to effectively bringing the pandanus extract to the target location, i.e. where it is needed. This is especially impressively shown by the unexpectedly high clinical cure rate in the application study (example 11: reduction of the SCORAD index by about 79% after 3 weeks of treatment). High cure rates like this can be compared to the effectiveness of the hydrocortisone therapy or to the effectiveness of stronger cortisone preparations and are therefore surprising for a plant extract of numerous components and unexpected for an expert. Thus, it is certainly surprising for an expert that a pandanus extract obtained by simple extraction procedures without any further expensive purification processes such as chromatography and without any further isolation of certain active agents from the extract shows such a strong therapeutic effect which is otherwise only known from pure active agents such as cortisone. Thus, according to the invention, formulations with an average drop diameter of less than 100 μm, preferably less than 50 μm, more preferably less than 1 μm and particularly preferably less than 500 nm are preferred as cream, ointment, gel or emulsion. The preference of such micro-formulations and nano-formulations also results from the examples 11 to 14.

### DESCRIPTION OF FIGURES

FIG. 1 shows a clear reduction of itching with neurodermatitis patients after only 21 days upon application of the inventive extract as defined in example 11.

FIG. 2 shows the course of the disease of neurodermatitis according to the SCORAD index after 3 weeks of application of the inventive extract as defined in example 11.

FIG. 3 shows the effect of pandanus extract on the LPS-induced TNF-α release in human monocytes.

FIG. 4 shows the effect of pandanus extract on the LPS-induced IL-6 release in human monocytes.

FIG. 5 shows the effect of pandanus extract on the LPS-induced PGE-2 release in human monocytes.

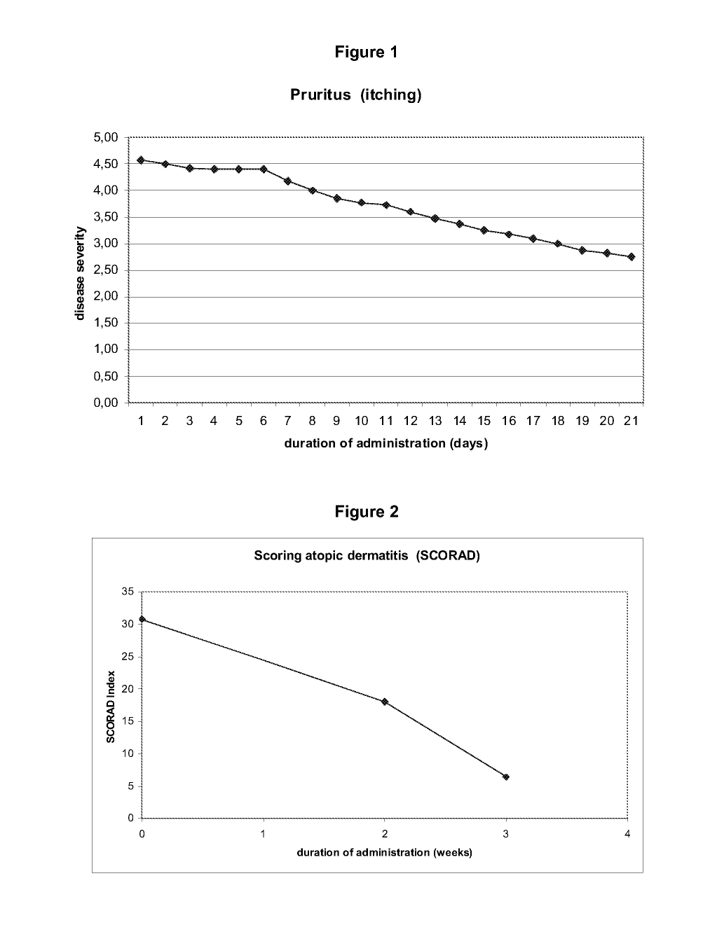

### EXAMPLES

### Example 1

### Production of Cold-Pressed Oil

The red fruit layer containing pips is stirred in a mixer preferably by adding water in order to separate the red pulp from the pips. The red fruit mush obtained is then pressed. After centrifugation and filtration, one obtains a clear, red, viscous oil (cold-pressed virgin oil; extract 1). The pips are washed with water and dried in the compartment drier at a temperature of max. 40° C. and stored for further processing.

### Example 2

### Production of the Oil by Extraction with Organic Solvents

The red fruit mush obtained as described above is extracted with a lipophilic solvent, e.g. hexane, pentane, cyclohexane, petroleum ether, heptane, tert-butyl methyl ether.

After drying (dehydration), centrifugation and filtration, one obtains the red, viscous oil.

### Example 3

### Production of Alcohol Extracts

The red fruit mush obtained as described above is extracted with a polar solvent, for example an alcohol, ether, acetone, THF, acetic acid ethyl ester and preferably methanol or ethanol.

After evaporation of the solvent, one obtains the alcoholic extract.

### Example 4

### Production of Alcohol Extracts from the Pips

The pips obtained as described above are crushed and extracted with 50% methanol water. After evaporation of the solvent, one obtains the extract of the pips (pip extract).

### Example 5

### Production of the Extract from the Dried Red Fruit Layer with Pips by Extraction with Organic Solvents

The red fruit part with pips is dried in a compartment drier at a temperature below 40° C. The water content of the dried material should be below 12%. The dried material is crushed and extracted with organic solvents such as methanol, ethanol, acetone, isopropyl alcohol or hexane, preferably with ethanol. One obtains a dark red, viscous oily product (oleosum).

Such extraction can of course be carried out with fresh fruit material, too.

### Example 6

### Production of the Aqueous Juice from the White/Pale Inner Fruit Layer

An aqueous phase is obtained from the white layer, i.e. the inner pale pulp, by direct pressing. Stabilizers such as ascorbic acid or other antioxidants can be added to the juice in order to prevent oxidation due to atmospheric oxygen. The juice can be directly used or dried for later use.

### Example 7

### Production of Alcohol Extracts from the Inner Pale Fruit Part

The white pale fruit part is cut into slices of about 4×4×2 cm and dried in the compartment drier. The pieces are crushed and extracted with 50% methanol water or 50% ethanol water.

### Example 8

### Extract Production from the Red Pulp with Supercritical Carbon Dioxide (Tk=31° C., Pk=73.9 bar)

The red fruit mush is mixed with the liquid carbon dioxide in the extraction container (90 bar, 8 kg CO₂/h over 4 h). The solvent loaded with plant ingredients is pumped into the separation container with a pump. On the way there, the pressure is reduced to subcritical conditions with a restriction valve. The solubility decreases and the released lipophilic substances fall out. In the separation container, the lipophilic substances (oil fraction) are separated from the gas. The gas is re-compressed to fluid and re-used. By adding different concentrations of entrainers (e.g. methanol or ethanol), medium-polar and polar substances may be gently extracted.

### Example 9

### Preparation of the Extracts

After thorough washing of the fruit with water, the outer red layer is separated from the inner white layer. The pulp part of the red layer is separated from the pips by shaking and/or rubbing.

The red pulp part is homogenized, pressed and centrifugated. After centrifugation, the oil phase is separated (extract 1). The residue is first extracted with n-hexane (extract 2) and then with methanol (extract 3).

The pips are crushed and extracted with 50% methanol (extract 4).

An aqueous phase is obtained from the white layer by direct pressing (extract 5). The residue after pressing is dried and extracted with 50% methanol (extract 6).

All steps of the procedure are carried out below 40° C.

### Example 10

### Production of Topical Formulations

Unless otherwise specified, the amounts indicated are percent by weight in relation to the weight and total amount and/or total weight of the preparation. The formulations described are suitable as cosmetic as well as pharmaceutical formulations. With regard to the pharmaceutical formulations, the proportion of pandanus extract is usually higher, preferably twice as high, than that of the cosmetic formulations.

Formulation 1:

| No. | Ingredients | Proportion (% by weight) |
|---|---|---|
| 1 | Lecithin | 4.0 |
| 2 | Extract according to example 5 | 1.0 |
| 3 | Glycerin | 5.0 |
| 4 | Propylene glycol | 16.0 |
| 5 | Medium-chain triglycerides | 3.0 |
| 6 | Lanolin | 10.0 |
| 7 | Purified water | ad 100 |

Formulation 2:

| No. | Ingredients | Proportion (% by weight) |
|---|---|---|
| 1 | Extract according to example 8 | 2.0 |
| 2 | Zinc oxide | 10.0 |
| 3 | Glycerin | 6.0 |
| 4 | Viscous paraffin | 30.0 |
| 5 | White vaseline | 20.0 |
| 6 | Bleached wax | ad 100 |

Formulation 3:

| No. | Ingredients | Proportion (% by weight) |
|---|---|---|
| 1 | Extract according to example 2 | 2.0 |
| 2 | Dexpanthenol | 1.5 |
| 3 | Miglyol | 5.0 |
| 4 | Purified water | 5.0 |
| 5 | Basis cream DAC | ad 100 |

Formulation 4:

| No. | Ingredients | Proportion (% by weight) |
|---|---|---|
| 1 | Extract according to example 3 | 3.0 |
| 2 | Medium-chain triglycerides | 5.0 |
| 3 | Polysorbate 60 | 8.0 |
| 4 | Propylene glycol | 6.0 |
| 5 | Groundnut oil | 10.0 |
| 6 | Glycerin | 10.0 |
| 7 | Vaselinum album | ad 100 |

Formulation 5:

| No. | Ingredients | Proportion (% by weight) |
|---|---|---|
| 1 | Extract according to example 4 | 8.0 |
| 2 | Urea | 4.0 |
| 3 | Lactic acid | 1.0 |
| 4 | Potassium sorbate | 0.15 |
| 5 | Purified water | 35.0 |
| 6 | Lanolin alcohol ointment | ad 100 |

Formulation 6:

| No. | Ingredients | Proportion (% by weight) |
|---|---|---|
| 1 | Extract according to example 7 | 6.0 |
| 2 | Medium-chain triglycerides | 4.0 |
| 3 | Basis cream DAC | ad 100 |

Formulation 7:

| No. | Ingredients | Proportion (% by weight) |
|---|---|---|
| 1 | Extract 1 and extract 4 (80:20) | 4.0 |
| 2 | Unguentum emulsificans aquosum | 45.0 |
| 3 | Purified water | ad 100 |

Formulation 8:

| No. | Ingredients | Proportion (% by weight) |
|---|---|---|
| 1 | Extract 2 and extract 5 (40:60) | 3.0 |
| 2 | Zinc oxide | 15.0 |
| 3 | Talcum | 15.0 |
| 4 | Glycerol | 30.0 |
| 5 | Propylene glycol | 10.0 |
| 6 | Purified water | ad 100 |

Formulation (O/W) 9:

| No. | Ingredients | Proportion (% by weight) |
|---|---|---|
| 1 | Extract according to example 3 | 2.0 |
| 2 | Glycerly stearate citrate | 1.0 |
| 3 | Polyethylene glycal(20)cetearyl ether | 10.0 |
| 4 | Dimethicone | 0.5 |
| 5 | Behenyl alcohol | 1.0 |
| 6 | Dicaprylyl carbonate | 3.0 |
| 7 | Tocopherol | 0.5 |
| 8 | Octyl dodecanol | 0.5 |
| 9 | Panthenol | 0.5 |
| 10 | Carbomer | 0.05 |
| 11 | Caprylic triglyceride | 1.0 |
| 12 | Methylparaben | 0.4 |
| 13 | Propylparaben | 0.3 |
| 14 | Sorbitol | 10.0 |
| 15 | Water | ad 100 |

Formulation (gel) 10:

| | | Proportion |
|---|---|---|
| No. | Ingredients | (% by weight) |
| 1 | Extract according to example 5 | 4.0 |
| 2 | Acrylate/C10-30 alkyl acrylate crosspolymer | 0.2 |
| 3 | Polyacrylic acid | 0.10 |
| 4 | Xanthan gum | 3.0 |
| 5 | Cetearyl alcohol | 4.0 |
| 6 | C12-15 alkyl benzoate | 3.0 |
| 7 | Caprylic/capric triglyceride | 5.0 |
| 8 | Cyclic dimethyl polysiloxane | 1.0 |
| 9 | Isopropyl | 3.0 |
| 10 | Sodium hydroxide | q.s. |
| 11 | Water | ad 100 |

Formulation (anhydrous formulation) 11:

| No. | Ingredients | Proportion (% by weight) |
|---|---|---|
| 1 | Extract according to example 8 | 4.0 |
| 2 | Polyglyceryl-3-diisostearate | 3.5 |
| 3 | Glycerin | 14.0 |
| 4 | Polyglyceryl-2-dipolyhydroxystearate | 3.5 |
| 5 | Miglycol | 6.0 |
| 6 | Propylene glycol | 2.0 |
| 7 | Sunflower oil | 4.0 |
| 8 | Vaselinum album | ad 100 |

Formulation (O/W) 12:

| | | Proportion |
|---|---|---|
| No. | Ingredients | (% by weight) |
| 1 | Extract according to example 1 + 4 (80:20) | 6.0 |
| 2 | Glyceryl stearate | 3.0 |
| 3 | PEG-100 stearate | 0.75 |
| 4 | Behenyl alcohol | 2.0 |
| 5 | Caprylic/capric triglyceride | 8.0 |
| 6 | Octyl dodecanol | 5.0 |
| 7 | C12-15 alkyl benzoate | 3.0 |
| 8 | Magnesium sulfate | 0.8 |
| 9 | Ethylene diamine tetraacetic acid | 0.1 |
| 10 | Water | ad 100 |

Formulation (O/W) 13:

| No. | Ingredients | Proportion (% by weight) |
|---|---|---|
| 1 | Extract according to example 4 | 4.0 |
| 2 | Glyceryl stearate | 2.0 |
| 3 | Sodium citrate | 0.174 |
| 4 | Citric acid | 0.086 |
| 5 | Glycerin | 10.0 |
| 6 | 1,3 butylene glycol | 0.5 |
| 7 | Hexanediol | 0.5 |
| 8 | Phenoxyethanol | 0.25 |
| 9 | Benzyl alcohol | 0.2 |
| 10 | Ethylparaben | 0.1 |
| 11 | Propylparaben | 0.1 |
| 12 | Phospholipon 90 G | 8.0 |
| 13 | Silicone oil, cyclic | 3.0 |
| 14 | Pentaerythrityl tetraisostearin | 3.0 |
| 15 | Triisostearin | 3.0 |
| 16 | BHT | 0.05 |
| 17 | PEG-40 stearate | 1.0 |
| 18 | Cetyl alcohol | 2.0 |
| 19 | Caprylic/capric triglyceride | 3.0 |
| 20 | Water | ad 100 |

Formulation (oil) 14:

| No. | Ingredients | Proportion (% by weight) |
|---|---|---|
| 1 | Extract according to example 1 | 3.0 |
| 2 | Jojoba oil | 5.0 |
| 3 | Cocoa butter | 0.05 |
| 4 | Macadamia nut oil | 5.0 |
| 5 | Medicinal white oil | 30.0 |
| 6 | Caprylic/capric triglyceride | 5.0 |
| 7 | Castor oil | 5.0 |
| 8 | Ethyl hexyl stearate | 10.0 |
| 9 | Tocopherol acetate | 0.1 |
| 10 | Tocopherol | 0.05 |
| 11 | Sunflower oil | ad 100 |

Formulation (W/O) 15:

| No. | Ingredients | Proportion (% by weight) |
|---|---|---|
| 1 | Extract according to example 6 | 2.0 |
| 2 | PEG-30 dipolyhydroxystearate | 0.5 |
| 3 | Lanolin alcohol | 1.5 |
| 4 | Paraffin oil | 10.0 |
| 5 | Vaseline | 6.0 |
| 6 | Hydrogenated cocoglycerides | 1.0 |
| 7 | Hydrogenated polyisobuten | 0.75 |
| 8 | Octyl dodecanol | 1.0 |
| 9 | Aluminium stearate | 0.3 |
| 10 | Dicaprylylcarbonate | 0.05 |
| 11 | Hydrogenated castor oil | 7.5 |
| 12 | Magnesium sulfate | 0.6 |
| 13 | Tetra sodium amino succinat | 0.6 |
| 14 | Caprylic/capric triglyceride | 2.5 |
| 15 | Methylparaben | 0.15 |
| 16 | Propylparaben | 0.4 |
| 17 | Glycerin | 5.0 |
| 18 | Water | ad 100 |

Formulation (cream) 16:

| | | Proportion |
|---|---|---|
| No. | Ingredients | (% by weight) |
| 1 | Extract according to example 7 | 5.0 |
| 2 | Glyceryl stearate | 2.5 |
| 3 | Stearyl alcohol | 2.0 |
| 4 | PEG-40 stearate | 1.0 |
| 5 | Cetyl alcohol | 2.0 |
| 6 | SHEA BUTTER | 2.0 |
| 7 | Carrageenan | 3.0 |
| 8 | Tapioca starch | 1.0 |
| 9 | Ethylhexyl methoxy cinnamate | 2.0 |
| 10 | Phenoxyethanol | 0.4 |
| 11 | C₁₂₋₁₅ alkylbenzoat | 3.0 |
| 12 | p-hydroxybenzol acid alkyl ester (paraben) | 0.4 |
| 13 | Aristoflex AVC (ammonium polyacryl | 0.2 |
| | dimethyltauride/vinylformamide copolymer) | |
| 14 | 2-isopropyl-5-methyl-cyclohexanecarbonyl-D- | 0.5 |
| | alanine ethyl ester | |
| 15 | Glycerin | 8.0 |
| 16 | EDTA | 0.2 |
| 17 | Water | ad 100 |

### Example 11

### Clinical Tests

### Principle and Methodology:

The aim of this test was to exactly test the inventive extract with regard to its tolerability and effectiveness according to clinical-dermatological test criteria. The patients could consult the dermatologist who accompanied the test every day with regard to objective and subjective changes of the skin.

At the beginning of the study, after 2 weeks of application and after 3 weeks of application, the dermatological examinations of the participants of the study were carried out including the assessment of the modified SCORAD.

Every test person then used the inventive extract at the affected spots of the skin twice per day for a total duration of three weeks. If necessary, the skin condition could be checked every day by the dermatologist accompanying the test.

The Used Inventive Extract:

The inventive extract used in the clinical study was produced according to example 5 and formulated as a cream with a concentration of 3% of pandanus extract.

TABLE 1
Composition of the nano-emulsion cream used for the application
study with neurodermatitis patients

| | % |
|---|---|
| Dipalmitoylphosphatidylcholine | 5.000 |
| Glycerol 85% | 1.500 |
| Dexpanthenol | 1.000 |
| Urea | 3.000 |
| Pandanus extract (example 5, ethanol extract) | 3.000 |
| Tocopherol acetate | 0.200 |
| Sodium benzoate | 0.150 |
| Aq. Dest. | 86.15 |
| | 100.00 |

### Patients:

The group of patients consisted of adult test persons with skin changes of neurodermatitis and in particular itching, reddening, desquamation or dryness. 20 persons with mild to medium neurodermatitis were examined.

| No. | Sex | Age | Diagnosis | Place of application |
|---|---|---|---|---|
| 1. | f | 65 | Neurodermatitis | Lower leg |
| 2. | f | 52 | Neurodermatitis | Upper arm |
| 3. | f | 57 | Neurodermatitis | Upper body |
| 4. | f | 52 | Neurodermatitis | Upper body |
| 5. | f | 30 | Neurodermatitis | Crook of the arm |
| 6. | f | 36 | Neurodermatitis | Upper body |
| 7. | f | 44 | Neurodermatitis | Upper body |
| 8. | m | 45 | Neurodermatitis | Leg |
| 9. | m | 42 | Neurodermatitis | Lower arms |
| 10. | f | 67 | Neurodermatitis | Hollow of the knee, |
| | | | | sole of a foot |
| 11. | f | 40 | Neurodermatitis | Upper body |
| 12. | f | 55 | Neurodermatitis | Crook of the arm |
| 13. | m | 45 | Neurodermatitis | Upper body |
| 14. | m | 48 | Neurodermatitis | Crook of the arm |
| 15. | f | 73 | Neurodermatitis | Hollow of the knee |
| 16. | m | 37 | Neurodermatitis | Lower arms |
| 17. | f | 31 | Neurodermatitis | Hollow of the knee |
| 18. | m | 53 | Neurodermatitis | Crook of the arm |
| 19. | f | 70 | Neurodermatitis | Upper arms |
| 20. | f | 68 | Neurodermatitis | Lower leg |

### Exclusion Criteria:

Acute organic disease
Pregnancy and lactation period
Existing sensitization to ingredients of the test products
Severe diseases
Application of preparations and care products containing the active agent until up to 4 weeks prior to the beginning of the tests
Intake of medication for neurodermatitis/atopy (glucocorticoids, antiallergenics, topical immunmodulators etc.)

### Carrying Out the Tests:

Only participants with whom no other skin changes requiring treatment were found were included in the test group.

At the beginning of the study, after 2 weeks of application and after 3 weeks of application, a dermatological examination of the participants of the study including the survey of the medical assessment sheet took place.

The inventive extract was given to the subjects with the application instruction of at least twice daily application. Moreover, the subjects were instructed not to take any other preparations during the time of the application test.

### Atopy Score (Modified According to SCORAD):

This index serves for the qualitative and quantitative assessment of the degree of severity of the atopic eczema, modified SCORAD (=severity scoring of atopic dermatitis).

The standardized assessment is carried out with the following information:
A) Degree of severity of six typical morphological changes (degree 0-3, total value here: max. 10)
B) Proportion of the affected skin surface (%) with regard to the reddening/slough/excoriation
C) Subjective evaluation of itching on the basis of a visual analogue scale (degree 0-10; here: max. 7) and of the loss of sleep at night (degree 0-10, here: max. 3).

### For A)

Six typical morphological changes are classified:

Reddening of the skin

### Sloughs

Extensive infiltration of the skin with coarsening of the skin fields
Swelling/blisters (here: exclusion criterion)
Excoriation up to the corium (here: exclusion criterion)
Dryness (assessed at the skin parts NOT affected)
Criterion: not present=0/mildly present=1/medium=2/severe=3

The sum of the values can vary between 0 and 10, while the severity of oedema or papules has been defined as an exclusion criterion as an indication of an acute neurodermatitic skin condition with medical need for being treated. Excoriation up to the corium has been defined as an exclusion criterion within the frame of the initial examination. Excoriations in the course of the study were an indication for an interruption of the test for 2 days.

### For B)

If the total value of the SCORAD exceeds 50 due to an extensive severity, this also results in the exclusion of the subject.

### For C)

The assessment of the itching >7 and of the loss of sleep >3 also leads to exclusion since the medical need for being treated and possible artifacts must be assumed.

The maximum overall value as modified SCORAD according to Dermatest amounts to 50 as an indication of a medium severity of the atopic symptoms. Calculation of the index: 7A/2+B/5+C.

### Dermatological Examinations:

### 1. Prior to the Beginning of the Application Test

All 20 participants of the study showed either mild flushes, desquamation or dryness. No other pathologic skin changes were found in any form.

### 2. During the Application Test

None of the 20 participants of the study showed any pathologic skin changes in the course of the three weeks of the application tests. No test interruptions or even treatments by dermatologists were necessary.

### 3. After the End of the Application Test

In the dermatological final examination after the end of the test, no additional pathologic skin changes were observed with the 20 participants of the study. The inventive extract was well tolerated and did not lead to any undesirable skin changes with any of the test persons.

Dermatological Assessment Criteria Registered with the Modified SCORAD:

1. Oedema/papules
2. Erythema
3. Slough formation
4. Excoriation
5. Lichenification
6. Dryness (xerosis)
7. Intensified itching
8. Loss of sleep

### Subjective Assessment Criteria:

1. Severity of the disease
2. Erythema
3. Dryness
4. Itching
5. Effectiveness of the test preparation

The assessment is made by the subjects within the frame of the daily documentation in the subjects' diaries. The scale covers the area 0-7; 0=not present, 7=extensively present.

### Assessment of the Test Results:

A total of 20 participants in the study tolerated the inventive extract without any adverse effects whatsoever in the three-week application test carried out in accordance with dermatological-clinical criteria. There was no case of undesirable or even pathologic skin changes. After application of the inventive extract over 14 days, the average SCORAD value was reduced from 30.79 to 18.04. This corresponds to a reduction by −41.40% compared to the initial value.

After application of the inventive extract over 21 days, the average SCORAD value of the participants of the study was reduced from 30.79 to 6.4. This corresponds to a reduction of the symptoms by −79.23% compared to the initial value.

The decrease of the atopy index must therefore be classified as excellent.

### Example 12

### Experience Report with Pandanus Extract Micro-Emulsions without Skin Care Additives

Pandanus extract micro-emulsion without further addition of the skin care substances (composition see table 2)

The application of pandanus extract micro-emulsion has been observed with three subjects with atopic eczema and symmetrically affected spots. The affected spot was treated twice daily, either with care lotion (free choice) or with pandanus micro-emulsion (left-right comparison). The treatment and observation period was one week. The change of the skin symptoms of the disease, namely itching, was compared before and after the treatment as well as between the treatments. All 3 subjects had reported severe to medium itching conditions prior to the beginning of treatment. (Scale of the itching symptoms: severe-medium-mild-none)

TABLE 2
Composition of the micro-emulsion only with pandanus extract
and emulsifiers

| | % |
|---|---|
| Lipoid S 20 | 12.00 |
| Mygliol | 4.00 |
| Ethanolic pandanus extract (example 5) | 8.00 |
| Sodium benzoate | 0.15 |
| Aq. Dest. | 80.85 |
| | 100.00 |

After 1 week of treatment with the pandanus micro-emulsion, the subjects reported a strong decrease of the itching (mild conditions: 2/no conditions: 1 subject), while the utilization of the care lotion showed less relief in comparison to the application of pandanus extract (severe conditions: 1/medium conditions: 2 subjects).

Pandanus extract nano-emulsion without further addition of the skin care substances (composition see table 2)

### Example 13

### Experience Report with Pandanus Extract Nano-Emulsions without Skin Care Additives

The application of pandanus extract nano-emulsion has been observed with three subjects with atopic eczema and symmetrically affected spots. The affected spot was treated twice daily, either with care lotion (free choice) or with pandanus nano-emulsion (left-right comparison). The treatment and observation period was one week. The change of the skin symptoms of the disease, namely itching, was compared before and after the treatment as well as between the treatments.

All 3 subjects had reported severe to medium itching conditions prior to the beginning of treatment. (Scale of the itching symptoms: severe-medium-mild-none)

TABLE 3
Composition of the nano-emulsion only with pandanus
extract and emulsifiers

| | % |
|---|---|
| Dipalmitoylphosphatidylcholine | 6.00 |
| Mygliol | 8.00 |
| Ethanolic pandanus extract (example 5) | 3.00 |
| Sodium benzoate | 0.15 |
| Aq. Dest. | 82.85 |
| | 100.00 |

After 1 week of treatment with the pandanus nano-emulsion, the subjects reported a strong decrease of the itching (no conditions: 2 subjects, mild conditions: 1 subject), while the utilization of the care lotion showed less relief in comparison to the application of pandanus cream (medium conditions 2 subjects/severe conditions: 1 subject).

The pandanus nano-emulsion cream seems to be more effective than the pandanus micro-emulsion cream.

### Example 14

### Pandanus Extract for Neurodermatitis

Production of Emulsions with Different Particle Sizes (Emulsion, Micro-Emulsion, Nano-Emulsion):

### Ingredients of the Emulsion Formulation:

| No. | Ingredients | Proportion (% by weight) |
|---|---|---|
| 1 | Ethanolic pandanus extract (example 5) | 3.0 |
| 2 | Lipoid S 100 | 5.0 |
| 3 | Miglycol | 4.0 |
| 4 | Methylparaben | 0.4 |
| 5 | Propylparaben | 0.3 |
| 6 | Carrageenan | 4.0 |
| 7 | Water | ad 100 |

Lipoid S 100 and water are mixed and stirred while being heated at approx. 50-60° C. until homogeneity is reached. Afterwards, carrageenan and then the other substances are added. The drop size of the emulsion was >50 μm (measured with the PSS Accusizer).

### Micro-Emulsion Formulation:

The micro-emulsion in the composition according to table 2 is produced with Ultra-Turrax on the basis of the production method described above.

The drop size was between 1 and 50 μm (measured with the PSS Accusizer).

### Nano-Emulsion Formulation

The ingredients as indicated in table 3 were pre-emulsified with Ultra-Turrax while being heated. The mixture was then homogenized with a high-pressure homogenizer at 700 bar and 4 cycles (Avestin Emulsiflex). The drop size was determined with PSS Nicomp.

The nano-formulation contained an average drop size between 100 and 400 nm.

### Examination of Neurodermatitis Patients:

A group of three subjects suffering from neurodermatitis was treated with the conventional emulsion for a period of 1 week.

Another group of three neurodermatitis subjects received the same amount of the micro-formulation over the same period of time and a third group of three neurodermatitis subjects received the nano-formulation.

The affected spot was treated twice per day. The treatment and observation period was one week. The change of the skin symptoms of the disease, namely itching, was compared before and after the treatment with different formulations. All subjects had reported severe to medium itching conditions prior to the beginning of treatment. (Scale of the itching symptoms: severe/4-medium/3-mild/2-none/1)

The group which received the macro-formulation shows an improvement of the neurodermatitis but showed the worst result in total. The groups which received the micro-formulation and nano-formulation achieved considerably better results; the nano-formulation, however, seems to result in a slightly better improvement than the micro-formulation.

Changes of the Skin Symptoms of Neurodermatitis Before and after the Twice-Daily Treatment Over One Week with Pandanus Extract in Different Formulations:

| Subjects | Macro-formulation | Micro-formulation | Nano-formulation |
|---|---|---|---|
| 1 | 4 → 3 | | |
| 2 | 4 → 3 | | |
| 3 | 3 → 3 | | |
| 4 | | 4 → 2 | |
| 5 | | 3 → 2 | |
| 6 | | 4 → 3 | |
| 7 | | | 3 → 2 |
| 8 | | | 4 → 1 |
| 9 | | | 4 → 2 |

Scale of the itching symptoms: severe (4) - medium (3) - mild (2) - none (1)

### Example 15

### Effect of Pandanus Extract on the LPS-Induced TNF-α Release in Human Monocytes

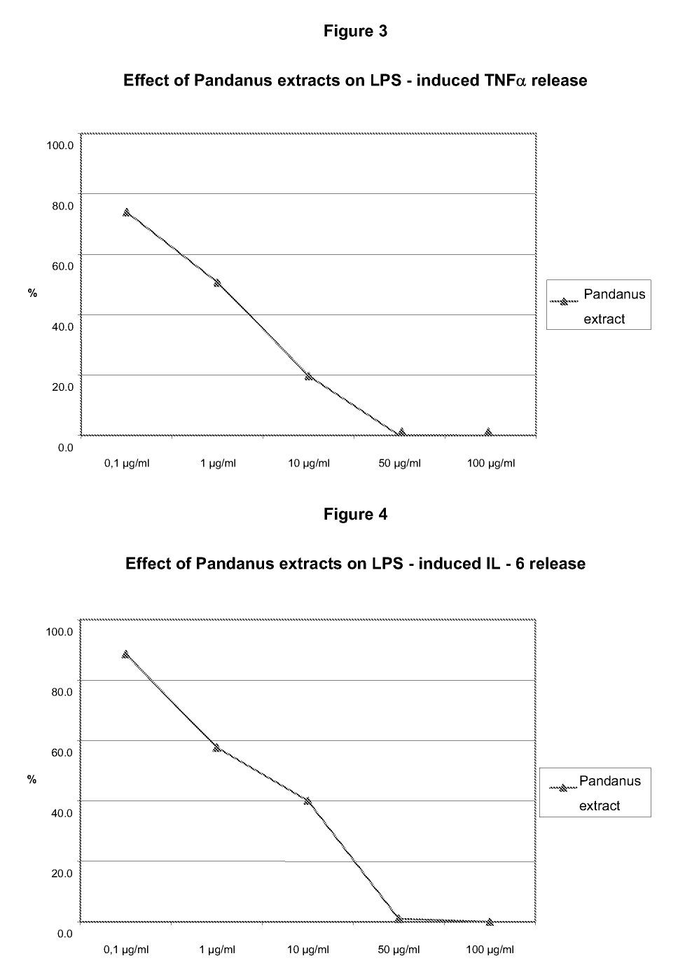

For the test, human monocytes of three different donors were respectively stimulated with LPS (10 ng/ml) in the absence and presence of pandanus extract in different concentrations. The results are summarized in FIG. 3 and show a 100% TNF-α release after stimulation with LPS without pandanus extract and a decreasing TNF-α release depending on the pandanus extract concentration used (n=3). The IC-50 amounts to <10 μg/ml.

### Example 16

### Effect of Pandanus Extract on the LPS-Induced IL-6 Release in Human Monocytes

For the test, human monocytes of three different donors were respectively stimulated with LPS (10 ng/ml) in the absence and presence of pandanus extract in different concentrations. The results are summarized in FIG. 4 and show a 100% IL-6 release after stimulation with LPS without pandanus extract and a decreasing IL-6 release depending on the pandanus extract concentration used (n=3). The IC-50 amounts to <10 μg/ml.

### Example 17

### Effect of Pandanus Extract on the LPS-Induced PGE-2 Release in Human Monocytes

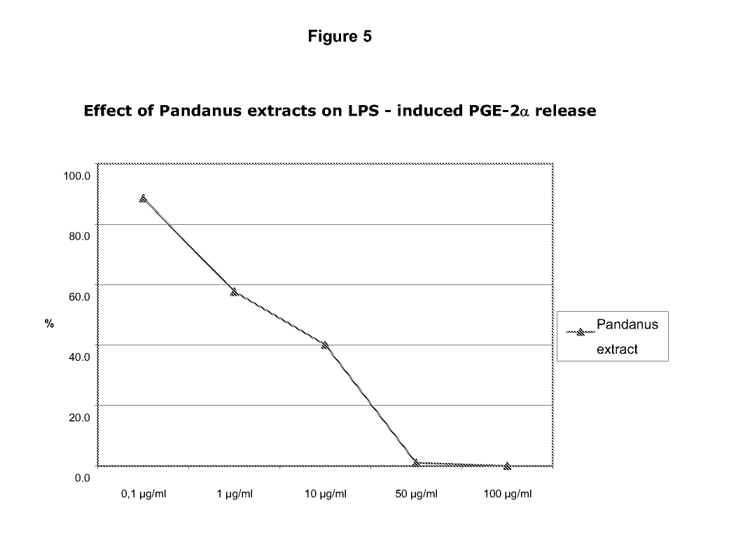

For the test, human monocytes of three different donors were respectively stimulated with LPS (10 ng/ml) in the absence and presence of pandanus extract in different concentrations. The results are summarized in FIG. 5 and show a 100% PGE-2 release after stimulation with LPS without pandanus extract and a decreasing PGE-2 release depending on the pandanus extract concentration used (n=3). The IC-50 amounts to <50 μg/ml.

### Example 18

### Experience Report with Pandanus Extract Nano-Emulsions for Prophylaxis for Neurodermatitis

The application of pandanus extract nano-emulsions for prophylaxis for neurodermatitis was observed with three subjects with atopic eczema and symmetrically affected spots. After a period of one month for recording the initial symptoms, the subjects used the pandanus extract nano-emulsion twice a day over a period of five months. The comparison of the symptoms reported by the patients with the initial symptoms served as a primary end point. All 3 subjects had reported severe to medium itching conditions prior to the beginning of treatment (scale of the itching symptoms: severe-medium-mild-none). After two weeks of treatment with the pandanus extract nano-emulsion, the subjects reported a considerable decrease of itching (no conditions: 3 subjects). The subjects remained mostly free of conditions over the complete duration of the study of five months and only reported short phases of mild itching.

TABLE 4
Composition of the nano-emulsion cream used for
prophylaxis for neurodermatitis

| | % |
|---|---|
| Dipalmitoylphosphatidylcholine | 5.000 |
| Glycerol 85% | 1.500 |
| Dexpanthenol | 1.000 |
| Urea | 3.000 |
| Pandanus extract | 3.000 |
| Tocopherol acetate | 0.200 |
| Sodium benzoate | 0.150 |
| Aq. Dest. | 86.15 |
| | 100.00 |

### Example 19

### Experience Report with Pandanus Extract Nano-Emulsions without Skin Care Additives

The application of pandanus extract nano-emulsions was observed with five subjects with psoriasis and symmetrically affected spots. A part of the affected spots was treated with a lotion of free choice and the other spots were treated with pandanus nano-emulsion twice a day (left-right comparison). As a lotion, the five subjects chose an aloe vera cream with 45% aloe in the following composition: 45% aloe vera, menthol, eucalyptus oil, wintergreen oil, jojoba oil, apricot kernel oil and sesame oil. The treatment and observation period was one week. The change of the skin symptoms of the disease, namely itching, was compared before and after the treatment as well as between the treatments. All 5 subjects had reported severe to medium itching conditions prior to the beginning of treatment (scale of the itching symptoms: severe-medium-mild-none).

TABLE 5
Composition of the nano-emulsion only with pandanus
extract and emulsifiers

| | % |
|---|---|
| Dipalmitoylphosphatidylcholine | 6.00 |
| Mygliol | 8.00 |
| Ethanolic pandanus extract (example 5) | 3.00 |
| Sodium benzoate | 0.15 |
| Aq. Dest. | 82.85 |
| | 100.00 |

After 1 week of treatment with the pandanus nano-emulsion, the subjects reported a strong decrease of the itching (mild conditions: 4 subjects, no conditions: 1 subject), while the utilization of the aloe vera lotion showed less relief in comparison to the application of pandanus cream (medium conditions: 5 subjects). In addition to this, all five subjects reported that the side treated with pandanus extract showed an improved skin condition.

### Example 20

### Experience Report with Pandanus Extract Nano-Emulsions for Prophylaxis for Psoriasis

The application of pandanus extract nano-emulsions for prophylaxis for psoriasis was observed with three subjects with symmetrically affected spots. After a period of one month for recording the initial symptoms, the subjects used the pandanus extract nano-emulsion twice a day over a period of five months. The comparison of the symptoms reported by the patients with the initial symptoms served as a primary end point. All 3 subjects had reported severe to medium itching conditions prior to the beginning of treatment (scale of the itching symptoms: severe-medium-mild-none). After two weeks of treatment with the pandanus extract nano-emulsion, the subjects reported a considerable decrease of itching (no conditions: 2 subjects, mild conditions: 1 subject). The subjects reported shorter phases of itching over the further course of the study but this could not reach the length and intensity of the initial symptoms any more.

TABLE 6
Composition of the nano-emulsion cream used
for prophylaxis for psoriasis

| | % |
|---|---|
| Dipalmitoylphosphatidylcholine | 5.000 |
| Glycerol 85% | 1.500 |
| Dexpanthenol | 1.000 |
| Urea | 3.000 |
| Pandanus extract | 3.000 |
| Tocopherol acetate | 0.200 |
| Sodium benzoate | 0.150 |
| Aq. Dest. | 86.15 |
| | 100.00 |

1. Utilization of an extract gained from the fruit of the species Pandanus conoideus for the production of a topical formulation for the treatment and prophylaxis of diseases of the skin and mucous membrane in the area of the nose, eyes and/or ears as well as for smoothing the skin and increasing the skin's hydration, with the topical formulation containing a micro-emulsion or a nano-emulsion or consisting of a micro-emulsion or a nano-emulsion.

2. Utilization according to claim 1, with the diseases of the skin being dry skin, neurodermatitis, dermatitis, eczema, psoriasis, metabolic disorders of the skin, acne, damaged skin and symptoms of aging.

3. Utilization according to claim 1, with the topical formulation containing a carrier system in the form of an oil-in-water emulsion or a water-in-oil emulsion.

4. Utilization according to claim 1, with the extract being obtained from the red outer pulp and/or the white pale inner pulp and/or from the pips.

5. Utilization according to claim 4, with the extract being obtained from the red outer pulp with the pips or only from the red pulp by direct pressing without any chemical additives.

6. Utilization according to claim 4, with the extract being obtained from the red outer pulp with the pips or only from the red pulp by extraction with organic solvents such as methanol, ethanol, propanol, isopropyl, acetone, ethyl acetate, hexane.

7. Utilization according to claim 4, with the extract from the pips being a hydrophilic extract which is obtained by separation and crushing of the pips and extraction of the crushed pips with a hydrophilic solvent.

8. Utilization according to claim 4, with the extract from the white pale inner pulp being a hydrophilic extract which is obtained by direct pressing of the separated white pale inner pulp or by drying and crushing of the separated white pale inner pulp and subsequent extraction of the dried crushed white pale inner pulp with a hydrophilic solvent.

9. Utilization according to claim 4, with the extract being obtained from the red outer pulp and/or from the white pale inner pulp and/or the pips with supercritical fluids.

10. Utilization according to claim 1, with the topical formulation being given in the form of an emulsion formulation, ointment, paste, cream or gel.

11. Cosmetic remedy for application in cases of dry skin, metabolic diseases of the skin, acne, damaged skin, symptoms of aging as well as for smoothing the skin and increasing the skin's hydration which contains an extract or extracts from the fruit of the species Pandanus conoideus.

12. Cosmetic remedy according to claim 11, with the cosmetic remedy being given in the form of an emulsion formulation, ointment, paste, cream or gel.

13. Cosmetic remedy according to claim 11, with the cosmetic remedy being a micro-emulsion or a nano-emulsion or consisting of a micro-emulsion or a nano-emulsion.

14. Cosmetic remedy according to claim 12, with the cosmetic remedy being a micro-emulsion or a nano-emulsion or consisting of a micro-emulsion or a nano-emulsion.
